(19) United States
(12) Patent Application Publication
(10) Pub. No.: US 2021/0370130 A1
(43) Pub. Date: Dec. 2, 2021

(54) ALTITUDE SIMULATOR AND ASSOCIATED CONTROL SYSTEMS AND METHODS

(71) Applicant: MOUNTAIN AIR HEALTH, INC., Edwards, CO (US)

(72) Inventors: JONATHAN JONIS, BOULDER, CO (US); MATTHEW NUNN, LEEDON HEIGHTS (SG)

(21) Appl. No.: 17/331,948

(22) Filed: May 27, 2021

Related U.S. Application Data
(60) Provisional application No. 63/031,461, filed on May 28, 2020.

(51) Int. Cl. A63B 23/18; A61M 16/00; A61G 10/02

(52) U.S. Cl. CPC A63B 23/18; A61M 16/0045; A63B 2225/50; A61G 10/023; A63B 2225/20; A61M 16/024

(57) ABSTRACT

An altitude simulator controls a percentage of oxygen within an enclosed environment to simulate altitude. A controller of the altitude simulator determines a set altitude to be simulated within the enclosed environment and determines an oxygen percentage setpoint based upon the set altitude. The altitude simulator senses, at intervals, environmental parameters of the enclosed environment. The altitude simulator may determine, from the environmental parameters, an oxygen percentage value indicative of a percentage of oxygen in air of the enclosed environment. The altitude simulator feeds hypoxic air into the enclosed environment from an air unit and controls a ventilation fan to maintain the oxygen percentage value at the oxygen percentage setpoint.

### RELATED APPLICATION

This Patent Application claims priority to U.S. Patent Application No. 63/031,461, filed on May 28, 2020, and titled “Altitude Simulator and Associated Control Systems and Methods,” entirety of which is incorporated herein by reference

### BACKGROUND

Hypoxic simulators, or altitude simulators, operate to simulate a high-altitude environment by manipulating the oxygen (O₂) percentage and other air conditions within a given area for the purpose of improving a user's cardio performance or for the user to lose weight. Current altitude simulators are manually controlled, which provides inferior control of the simulation because there is often a 1 to 2 hour lag between an adjustment in the hypoxic air flow rate and the stabilization of the O₂ levels inside a tent or other enclosure (hereinafter called tent). Also, if the user opens the tent to enter or exit, it changes the O₂ levels again and takes some time to stabilize again.

### SUMMARY

In one embodiment, an altitude simulator automatically controls an oxygen percentage in an enclosed environment. The altitude simulator includes a tent defining the enclosed environment and having an inlet. The altitude simulator also includes an air unit, air tubing having a first end coupled to the air unit and a second end positioned at the inlet, a ventilation fan, and a controller. The controller has: a communication module; an array of environmental sensors; a processor; and a memory storing machine readable instructions that, when executed by the processor, cause the processor to: determine, based on the array of environmental sensors, an oxygen percentage of air within the enclosed environment; and control a speed and direction of the ventilator fan to adjust the oxygen percentage within the enclosed environment.

In another embodiment, a method simulates altitude in an enclosed environment. The method includes determining, within a controller of an altitude simulator, a set altitude to be simulated within the enclosed environment; determining an oxygen percentage setpoint based upon the set altitude; sensing, at intervals, environmental parameters of the enclosed environment; determining, from the environmental parameters, an oxygen percentage value indicative of a percentage of oxygen in air of the enclosed environment; feeding hypoxic air into the enclosed environment from an air unit; and controlling a ventilation fan to maintain the oxygen percentage value at the oxygen percentage setpoint.

### BRIEF DESCRIPTION OF THE FIGURES

The foregoing and other features and advantages of the disclosure will be apparent from the more particular description of the embodiments, as illustrated in the accompanying drawings, in which like reference characters refer to the same parts throughout the different figures. The drawings are not necessarily to scale, emphasis instead being placed upon illustrating the principles of the disclosure.

FIG. 1 is a block diagram showing one example altitude simulator and associated control systems, in embodiments.

FIGS. 2A-2F are perspective views showing components of the altitude simulator of FIG. 1 coupled together, in embodiments.

FIGS. 3A-3D are perspective views showing additional example detail of the controller of the altitude simulator of FIG. 1, in embodiments.

FIGS. 4A-4C show example screenshots of the application running on a user device and controlling the altitude simulator of FIG. 1, in embodiments.

FIGS. 5A-5B are flowcharts illustrating one example method for controlling the altitude simulator of FIG. 1, in embodiments.

FIG. 6 is a flowchart illustrating one example process control method implemented within the controller of FIG. 1, in embodiments.

FIG. 7 is a flowchart illustrating one example method implemented by the user device of FIG. 1 to request data from an external server, in embodiments.

### DETAILED DESCRIPTION OF THE EMBODIMENTS

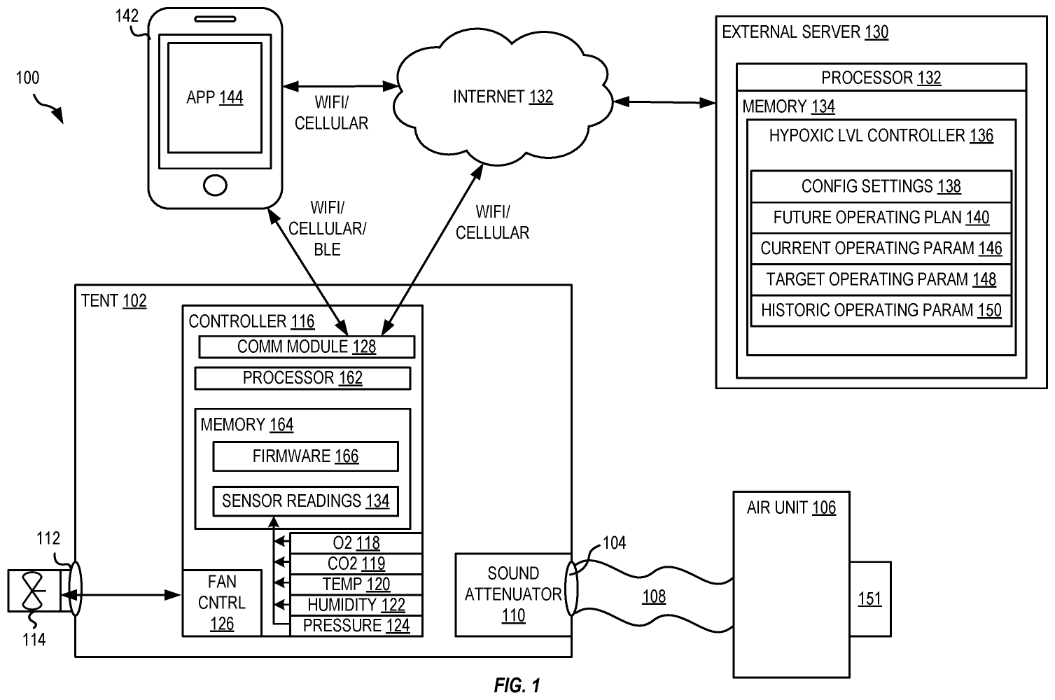

FIG. 1 is a block diagram showing one example altitude simulator 100 and associated control systems, in embodiments. Altitude simulator 100 includes a tent 102 with seals that allow air within tent 102 to be controlled, an air unit 106 (also known as an air separation unit, or an air handling unit), a ventilation fan 114, and a controller 116 that may control operation of ventilation fan 114 and air unit 106. The seals of tent 102 are not necessarily fully air-tight. In one example, panels of tent 102 are sealed with a zipper and/or a magnetic strip (each of which may be lined with silicone, rubber or other sealing material). Tent 102 includes an inlet 104 that may couple to air unit 106 via air tubing 108. Air unit 106 may be an off-the shelf, high-volume oxygen (O₂) concentrator with a small compressor that pushes air through a molecular sieve, from which hypoxic air comes out of a first side and hyperoxic air comes out of the other side.

Electric power supplied to air unit 106 may be controlled by a Wi-Fi enabled interlock 151, which, in certain embodiments, is an on/off power controller. Interlock 151 may be manually controlled (e.g., by a user) via a mobile application 144 running on a mobile device 142 (e.g., a smartphone, tablet computer, laptop computer, etc.) of the user. Interlock 151 may also be controlled using control signals generated by controller 116 and/or an external server 130. Interlock 151 may also operate as a safety mechanism that automatically turns off air unit 106 (via controller 116) when any sensor or system parameter is outside of expected values. In one embodiment, interlock 151 is positioned between an AC power cord of air unit 106 and the user's (e.g., home) AC power outlet. Controller 116 includes at least one processor 162 communicatively coupled with memory 164 storing firmware 166 that includes machine-readable instructions executable by the at least one processor 162 to implement functionality of controller 116 described herein.

Air tubing 108 may be a hypoxic air hose suitable for food/gas service (e.g., compliant to FDA, USDA, USP Class VI, NSF, 3A, and UL). In certain embodiments, altitude simulator 100 also includes a sound attenuator 110 that attaches to one end of air tubing 108 to reduce sound from air unit 106 entering tent 102. For example, sound attenuator 110 decreases noise transmission down air tubing 108 from an average of 62 dB(A) at lm to an average of 52 dB(A) at 1 m. Tent 102 also includes an integrated fan pocket 112, formed of mesh for example, for supporting a ventilation fan 114 that operates to regulate the amount of O₂ (and other environmental details) within tent 102 by either introducing ambient air from outside of tent 102, or extracting air from within tent 102 to help achieve an O₂ setpoint. Ventilation fan 114 is a variable-speed DC fan capable of operating in dual directions to pull or push air from/to the interior of tent 102.

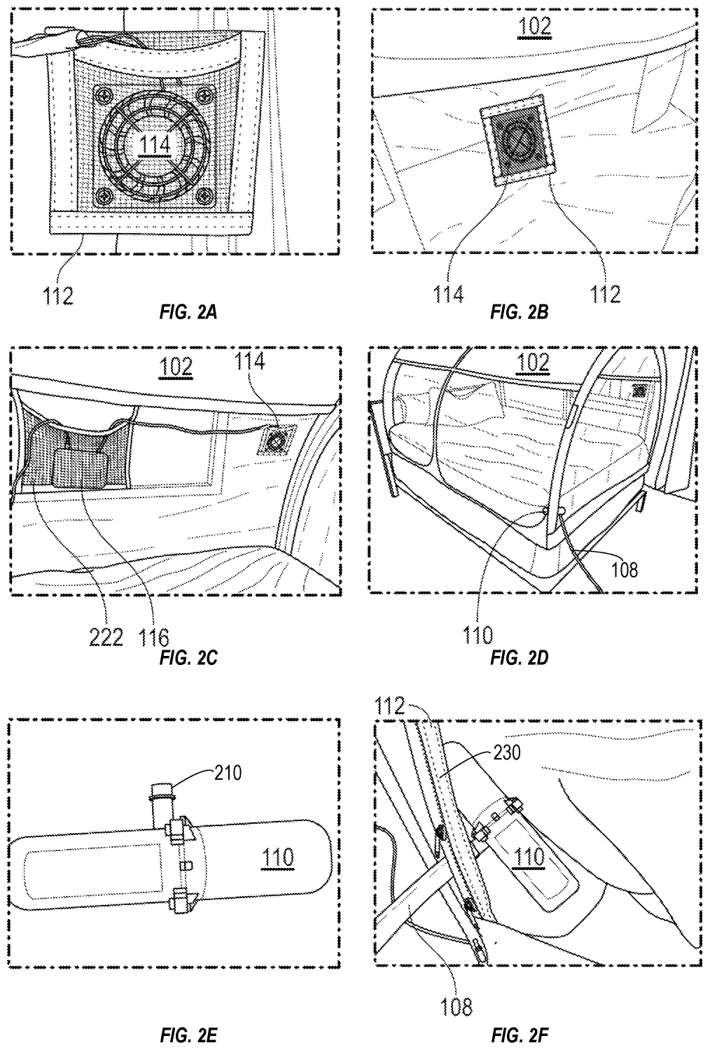

FIGS. 2A-2F are perspective views showing components of the altitude simulator of FIG. 1 coupled together. FIGS. 2A and 2B show ventilation fan 114 positioned within integrated fan pocket 112 of tent 102. FIG. 2C shows controller 116 positioned within an interior mesh pocket 222 of tent 102 and wired to ventilation fan 114. FIG. 2D shows sound attenuator 110 positioned within tent 102 and coupled to air tubing 108. FIG. 2E shows sound attenuator 110 in further example detail, including an inlet port 210 that couples with air tubing 108. FIG. 2F shows sound attenuator 110 positioned within tent 102 and air tubing 108 passing through a seal 230 of tent 102.

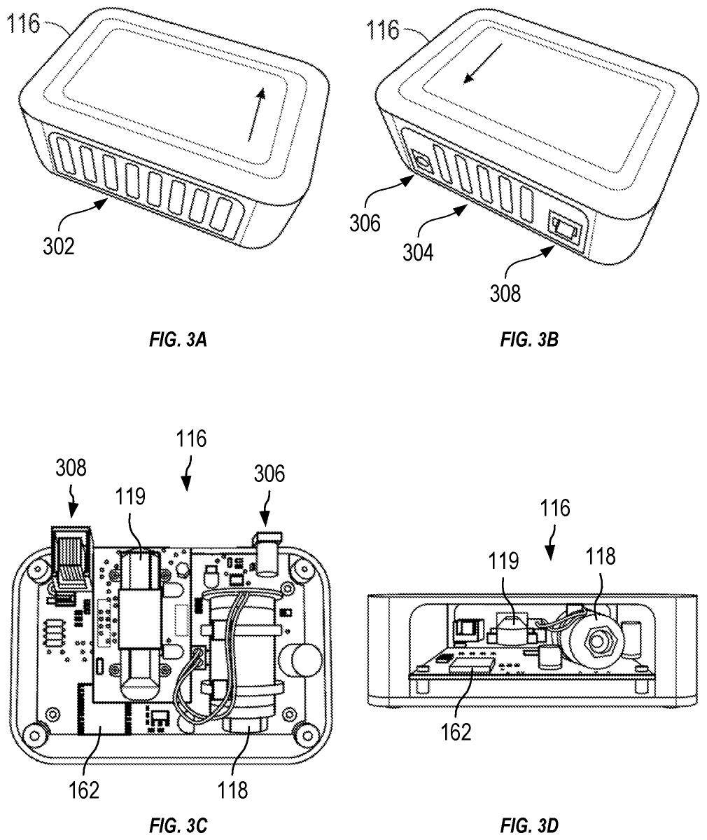

FIGS. 3A-3D are perspective views showing additional example detail of controller 116 of FIG. 1. FIGS. 3A and 3B show controller 116 with an air inlet 302 on a first side and an air outlet 304 on a second side opposite the first side, a DC power cable socket 306 and a ventilation fan cable socket 308. FIGS. 3C and 3D show example components housed within controller 116. FIGS. 1 through 3D are best viewed together with the following description.

Controller 116 is positioned within tent 102, such as in interior mesh pocket 222 (see FIG. 2C) that allows air within tent 102 to circulate through controller 116, to monitor environmental parameters within tent 102. To monitor environmental parameters, controller 116 uses one or more of an O₂ sensor 118, a carbon dioxide (CO₂) sensor 119, a temperature sensor 120, a relative humidity sensor 122, and a pressure sensor 124 that measures ambient barometric air pressure. Controller 116 also includes a fan controller 126 that controls operation of ventilation fan 114.

Controller 116 includes a wireless communication module 128 that allows controller 116 to wirelessly couple with one or both of a user device 142 and external server 130. Wireless communication module 128 may use any wireless data communication protocol, such as one or more of cellular (e.g., 2G, 3G, 4G, 5G, NB-IoT, etc.), Wi-Fi, Bluetooth, BLE, etc. For example, wireless communication module 128 may use one or both of cellular and Wi-Fi protocols to wirelessly couple controller 116 to external server 130, via the internet 132 for example. In certain embodiments, wireless communication module 128 may implement wired protocols through a wired interface as well as, or instead of, the wireless interface and wireless protocols.

In one example of operation, controller 116 may operate according to initial settings for O₂ control, altitude control, and data logging. The initial settings, for example, may auto-calibrate the O₂ sensor to a predefined value (e.g., 20.95% normoxia) upon each startup of controller 116 and in between startups of the air unit 106. This predefined value is selected, for example, because in the real-world, ambient air, regardless of altitude, has 20.95% O₂ content. At higher (real-world) altitudes, the O₂ % remains the same but the atmospheric pressure reduces so the body receives fewer molecules of O₂. Altitude simulator 100 produces a similar effect at normobaric conditions by reducing the O₂ % inside the tent. During operation of altitude simulator 100, the simulated altitude may cause a wide range of O₂ % within tent 102, but when altitude simulator 100 is not in operation, the O₂ % inside tent 102 always reverts to 20.95%, and thus, auto-calibration may be performed when altitude simulator 100 starts.

Controller 116 may implement an O₂ Proportional, Integral, Derivative (PID) control loop to determine, based on the initial settings, an initial setpoint for O₂ % and control the ventilation fan 114, within integrated fan pocket 112, to generate an airflow between inside and outside of tent 102 to dilute the hypoxic air inside tent 102 to achieve the O₂ % setpoint. Controller 116 may determine initial settings by using pressure sensor 124 to detect an ambient altitude as a starting point. Controller 116 may then control O₂ % within tent 102 to simulate a first period of use at 3000 feet above the ambient altitude starting point, and increase the simulated altitude by 1000 feet (e.g., each subsequent night) until a target simulated altitude of 10,000 feet above sea level is reached. A user may override these initial settings using their user device 142, as discussed below. In certain embodiments, controller 116 may include a maximum simulated altitude threshold that limits operation of altitude simulator 100. For example, controller 116 may include a maximum simulated altitude threshold that prevents tent 102 from achieving a simulated altitude above 14,000 feet above sea level.

Controller 116 may, at intervals, capture, as sensor reading 134 stored in memory 164, readings from one or more of O₂ sensor 118, CO₂ sensor 119, temperature sensor 120, relative humidity sensor 122, pressure sensor 124, and fan controller 126. Controller 116 may transmit sensor readings 134 to external server 130, for processing and analysis thereon. External server 130 is a digital computer that includes at least one processor 132 communicatively coupled with memory 134 storing a hypoxic level controller 136 that includes machine-readable instructions executable by the at least one processor 132 to implement functionality of external server 130 described herein. In certain embodiments, external server 130 is not included in altitude simulator 100, and functionality of hypoxic level controller 136 is implemented by controller 116.

Hypoxic level controller 136 of external server 130 may process the received sensor readings 134 to determine configuration settings 138 that are transmitted to controller 116 for use therein to create a specific environmental condition (e.g., simulated altitude level) within tent 102. Additional details for determining configuration settings 138 are discussed below.

In certain embodiments, hypoxic level controller 136 may receive the user's weight, input by the user using user device 142 for example, that is used to determine a future operating plan 140. Future operating plan 140 may define configuration settings for controller 116 that define use and control of one or more of tent 102, air unit 106, and ventilation fan 114 to help the user achieve a weight goal. The user may then sleep or spend an amount of time defined by future operating plan 140 in tent 102, whereby sustained time at the hypoxic levels created by controller 116, and defined by future operating plan 140, cause the user's body to function in a manner that achieves weight loss. Future operating plan 140 may further define a threshold level of altitude increase over a predetermined time (e.g., 3 days, 5 days, 1 week, 1 month, etc.) so that the user does not experience altitude sickness due to the simulated altitude.

The hypoxic level controller 136 addresses a plurality of potential problems associated with hypoxic level simulators. Potential problems include: (a) the air unit may produce too much hypoxic air for a small volume of the tent; (b) when a user (or two users) is (are) inside the tent, CO2 builds up, it gets stuffy, and body heat causes temperatures inside the tent to increase by as much as 5° F. (2.8° C.). This is especially so for two people in a queen-sized tent which only has an interior volume of 83 ft3 (2360 liters); and, (c) the user “ascends” (simulates increased altitude) too quickly, sleep quality suffers and they may experience symptoms of altitude sickness.

Hypoxic level controller 136 (and other components of altitude simulator 100) addresses these potential problems by, instead of controlling O₂ % within tent 102 by reducing the amount of hypoxic air going into tent 102 from air unit 106, hypoxic level controller 136 and controller 116 keep air unit 106 operating continuously (e.g., on maximum) to provide a continuous flow, and control ventilation fan 114 within integrated fan pocket 112 to operate as a separate automated ventilation system that dilutes the hypoxic air to achieve the desired altitude setpoint. This has the added benefit of purging CO₂ from tent 102 and keeps a fresh air flow to reduce stuffiness and build-up of heat. Additionally, controller 116 and/or hypoxic level controller 136 may be programmed to initially increase the altitude set-point over a 1-2 week period (or other time period) to avoid symptoms of altitude sickness.

In certain embodiments, user device 142 is coupled (wirelessly or via hardwire) to the controller 116 and/or the external server 130. For example, the user device 142 may couple to the controller 116 via Bluetooth Low Energy (BLE) connection. Mobile application 144, running on user device 142, may display data regarding current operating parameters 146 and target operating parameters 148. The current operating parameters 146 are received at the user device 142 from the controller 116 or external server 130. The target operating parameters 148 may be defined by user interaction with the user device 142 via the mobile application 144, and include one or more of target altitude, auto increase profile, and user contact details. The mobile application 144 may further display historical operating parameters 150, received at the user device 142 from the controller 116 or external server 130, defining prior statistics of use and conditions within the tent 102.

The user device 142 may be required to onboard with the controller 116, and the associated external server 130. To onboard, the User installs the mobile application 144 on the user device 142. Then the user, via the mobile application 144, registers an account associated with the external server 130, and provides user contact details. The user may scan a QR code (or input other ID information such as a serial number associated with the controller 116). This information is then passed to the external server 130 and stored in association with a user's profile. By onboarding the controller 116, the external server 130 may then pass the current operating parameters 146 and historical operating parameters 150 to the user device 142 for display to the user via mobile application 144. Furthermore, the user may interact with the user device 142 and the mobile application 144 to define the target operating parameters 148, which are passed to the external server 130 and used to determine the configuration settings 138 to control the controller 116, air unit 106, and tent 102.

Accordingly, the mobile application 144 may provide a variety of functions, including but not limited to: (a) detecting the controller 116 and AC power interlock 151 over Wi-Fi and connecting with them (similar to Sonos®, Roomba®, or other IoT device); (b) receiving an initial home Wi-Fi password for controller; (c) turning system 100 on/off and slowing down the ascent rate if user does not feel well; (d) receiving an altitude set-point manually entered by the user; (e) accessing (using a login with user id/password) a user profile that includes starting weight/height, personal data, and, optionally, payment info; (f) showing a line graph of user's altitude over a 24-hour period; (g) showing a line graph of user's altitude over a two week period; (h) receiving a user selection of how often to send email reports of logged parameters (daily, weekly, or monthly); (i) sharing, based on user directives, progress and data over common social media feeds such as Twitter/Facebook/Instagram/etc.; (j) purchasing, via interaction with the user, a monthly or annual service contract/data logging plan; (k) selecting, via interaction with the user, imperial or metric units; (l) selecting, via interaction with the user, a time zone; (m) changing, via user interaction, a password and/or logging out from the profile; (n) interfacing with Fitbit Wi-Fi scale and logging/displaying user weight vs. time; and (o) showing hours of operation and alerting the user to scheduled maintenance intervals (inlet filter change, interior filter change, and full service) by email.

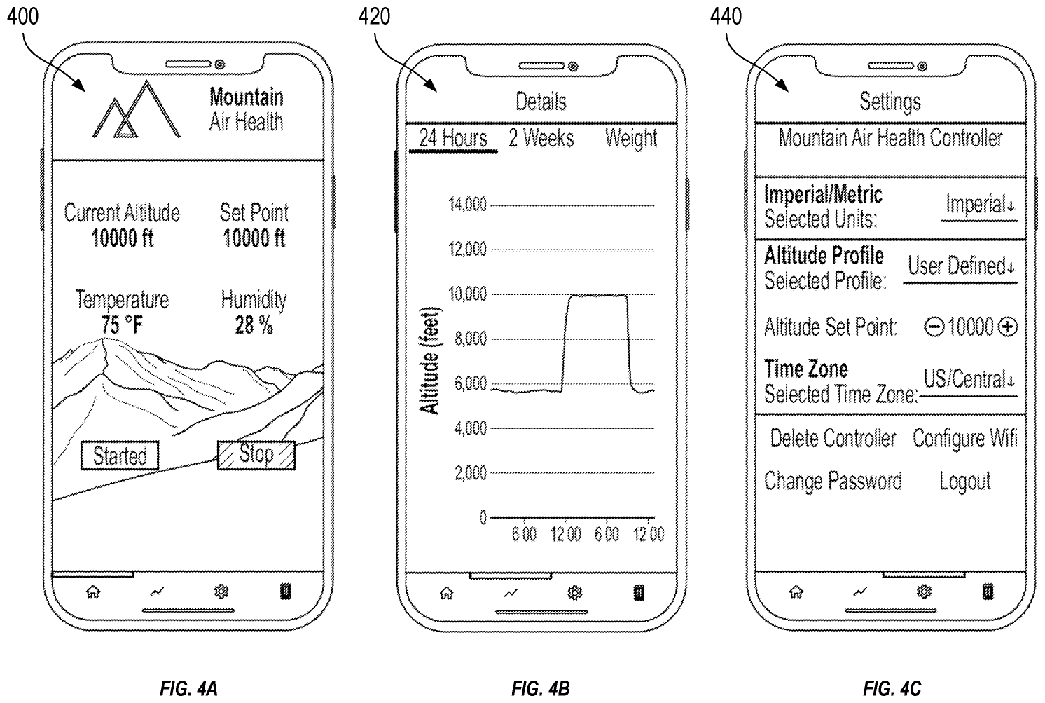

FIGS. 4A-4C are example screenshots of mobile application 144 running on user device 142. FIG. 4A shows a home page 400, presented by mobile application 144, that displays a current (simulated) altitude inside tent 102, an altitude set point, and a current temperature and humidity inside tent 102. In certain embodiments, home page 400 may also indicate a speed percentage (e.g., from zero to 100%) of ventilation fan 114. For example, a user may use the displayed ventilation fan 114 speed percentage to optimize the hypoxic air flow from air unit 106. If the speed percentage of ventilation fan 114 is less than 50%, the user should increase the hypoxic air flow from air unit 106 using a flow adjustment knob on air unit 106. This is to ensure adequate airflow into tent 102 and to lessen the uncomfortable increases in humidity, temperature, and CO₂ buildup inside tent 102 that may result from a person or persons being inside tent 102. If the speed percentage of ventilation fan 114 is greater than 80%, the user should decrease the hypoxic air flow from air unit 106 such that ventilation fan 114 does not make excessive noise and possibly prevent the user from sleeping well. FIG. 4B shows a second screen 420, presented by mobile application 144, that displays a graph of simulated altitude over time (24 hours in the example shown). FIG. 4C shows a third screen 440, presented by mobile application 144, that displays a settings screen that allows the user to configure mobile application 144.

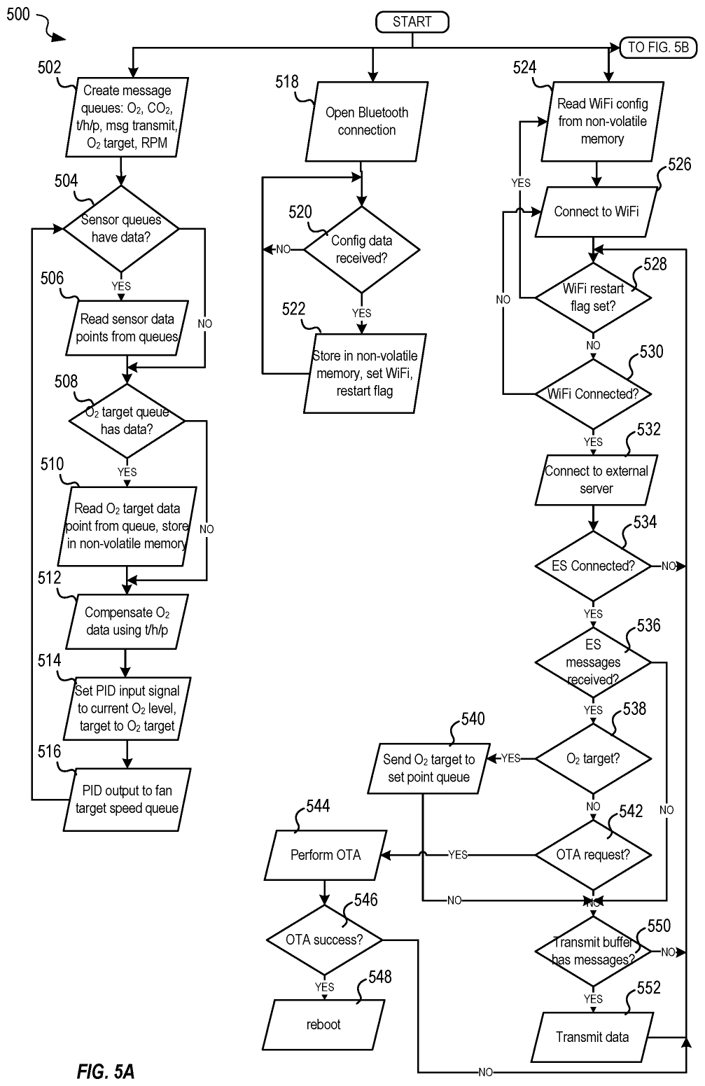

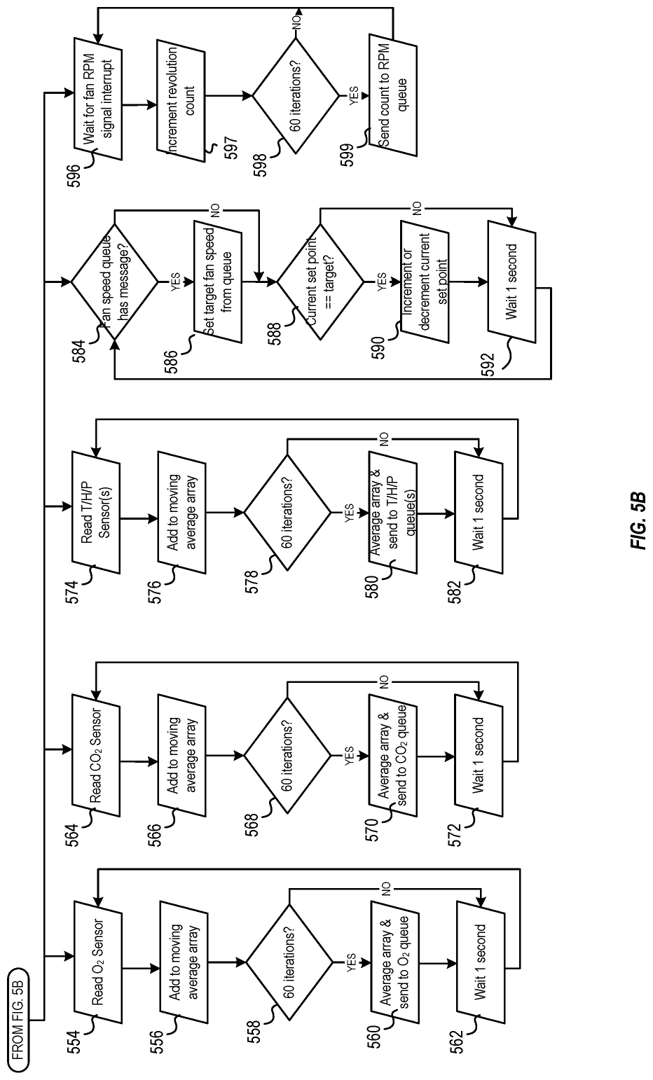

FIGS. 5A-B are flowcharts illustrating one example method 500 for altitude simulation and control thereof. Method 500 is implemented in one or more of controller 116, mobile application 144, and external server 130 of altitude simulator 100, for example, and may include any one or more (or any combination of) the blocks shown in FIG. 5. Each block in method 500 may be implemented via execution, by a processor associated with one or more components in altitude simulator 100, of one or more computer-readable instructions that when executed by the processor control the processor to implement the associated function described with respect to the given block.

In block 502, the controller 116 creates message queues for later transmission to external server 130 requesting information regarding O₂, CO₂, temperature, humidity, and atmospheric pressure from the respective sensors 118, 119, 120, 122, 124. Controller 116 also queues the O₂ target setpoint and ventilation fan 114 RPM setting.

Block 504 is a decision. If, in block 504, controller 116 determines that sensor queues have data (e.g., that the required data is stored in sensor readings 134), method 500 continues with block 506; otherwise, method 500 continues with block 508.

In block 506, controller 116 reads sensor data points from sensor readings 134 according to the queues.

Block 508 is a decision. If, in block 508, controller 116 determines that O₂ target setpoint has data, method 500 continues with block 510; otherwise, method 500 continues with block 512.

In block 510, controller 116 reads O₂ target setpoint from queue and stores in non-volatile memory of the controller 116. In block 512, controller 116 performs a compensation calculation to correct O₂ data using current temperature, humidity, and atmospheric pressure read from the associated sensors. For example, O₂ data read from O₂ sensor varies from actual O₂ levels because of ambient temperature, humidity, and atmospheric pressure and is therefore adjusted to correct for current conditions.

Controller 116 may include a PID control loop to regulate fan speed in order to control O₂ levels within tent 102. In block 514, controller 116 sets the process variable level to the current O₂ input signal and the target setpoint to the O₂ target. In block 516, controller 116 sends the PID control loop output to the fan target speed queue.

In block 518, controller 116 opens a Bluetooth Low Energy (BLE) connection and connects with mobile application 144 running on user device 142. Block 520 is a decision. If, in block 520, controller 116 determines that configuration data (altitude profile, altitude setpoint, time zone, and Wi-Fi login credentials) has been received, method 500 continues with block 522; otherwise, method 500 repeats block 520 until configuration data is received.

In block 522, controller 116 stores configuration data received from user device 142 in its non-volatile memory (e.g., memory 164). In certain embodiments, the configuration data is received via the external server 130 via a Wi-Fi connection established in steps 518-520.

In block 524, controller 116 reads Wi-Fi login credentials from its non-volatile memory (e.g., memory 164). In block 526, controller 116 connects to user's Wi-Fi network using credentials received in block 524. Block 528 is a decision. If, in block 528, controller 116 determines that a Wi-Fi restart flag set exists, method 500 continues with block 524; otherwise, method 500 continues with block 530.

Block 530 is a decision. If, in block 530, controller 116 determines that it has connected to the Wi-Fi network, method 500 continues with block 532; otherwise, method 500 continues with block 526.

In block 532, controller 116 connects to an account, associated with controller 116 and or the user of mobile device 142, on external server 130. Block 534 is a decision. If, in block 534, controller 116 determines that it has connected to external server 130, method 500 continues with block 536; otherwise, method 500 continues with block 528.

Block 536 is a decision. If, in block 536, controller 116 determines that messages from external server 130 have been received, method 500 continues with block 538; otherwise, method 500 continues with block 550.

Block 538 is a decision. If, in block 538, controller 116 determines that O₂ target has been received from external server 130, method 500 continues with block 540; otherwise, method 500 continues with block 542.

In block 540, the controller 116 sends O₂ target to set point queue. Block 542 is a decision. If, in block 542, controller 116 determines that an Over-The-Air (OTA) firmware upgrade request has been received from external server 130, method 500 continues with block 544; otherwise, method 500 continues with block 550.

In block 544, external server 130 transmits the firmware update OTA to controller 116 and controller 116 installs the firmware update OTA (e.g., into memory 164). Block 546 is a decision. If, in block 546, controller 116 and external server 130 confirm that the OTA update was successful, method 500 continues with step 548; otherwise, method 500 continues with block 528. In block 548, controller 116 reboots itself.

Block 550 is a decision. If, in block 550, controller 116 determines that its transmit buffer queue has outgoing messages, method 500 continues with block 552; otherwise, method 500 continues with block 528. In block 552, controller 116 transmits data from the transmit buffer queue to external server 130.

In block 554, controller 116 reads O₂ sensor output (either directly from the O₂ sensor 118, or from the sensor readings 134). In block 556, controller 116 adds O₂ sensor output to a moving average array in sensor readings 134. Block 558 is a decision. If, in block 558, controller 116 determines that there are 60 iterations (more or fewer iterations may be used without departing from the scope hereof) of O₂ sensor data output in sensor readings 134, method 500 continues with block 560; otherwise, method 500 continues with block 562.

In block 560, controller 116 calculates an average of the past 60 (or other quantity) O₂ sensor data outputs and sends the average to the O₂ queue. In block 562, controller 116 waits one second (or some other predetermined time period) before continues with block 554 (or other blocks within method 500). Blocks 554 through 562 repeat at a defined interval.

In block 564, controller 116 reads CO₂ sensor output (either directly from the CO₂ sensor 119, or from the sensor readings 134). In block 566, controller 116 adds CO₂ sensor output to a moving average array in sensor readings 134. Block 568 is a decision. If, in block 568, controller 116 determines that there are 60 iterations (more or fewer iterations may be used without departing from the scope hereof) of CO₂ sensor data output in sensor readings 134, method 500 continues with block 570; otherwise, method 500 continues with block 572. In block 570, controller 116 calculates an average of the past 60 (or other quantity) CO₂ sensor data outputs and sends the average to the CO₂ queue. In block 572, controller 116 waits one second (or some other predetermined period) before proceeding to block 564 (or other blocks within method 500). Blocks 564 through 572 thus repeat at a defined interval.

In block 574, controller 116 reads temperature/humidity/pressure (T/H/P) sensor(s) output (either directly from the temperature sensor 120, relative humidity sensor 122, and/or pressure sensor 124, or from the sensor readings 134). In block 576, controller 116 adds T/H/P sensor(s) output to a moving average array in sensor readings 134. Block 578 is a decision. If, in block 578, controller 116 determines that there are 60 iterations (more or fewer iterations may be used without departing from the scope hereof) of T/H/P sensor data output in sensor readings 134, method 500 continues with block 580; otherwise, method 500 continues with block 582. In block 580, controller 116 calculates an average of the past 60 (or other quantity) T/H/P sensor data outputs and sends the average to the T/H/P queue. In block 582, controller 116 waits one second (or some other predetermined time period) before proceeding to block 574 (or other blocks within method 500). Blocks 574 through 582 thus repeat at a defined interval.

Block 584 is a decision. If, in block 584, controller 116 determines that fan speed queue has a message (e.g., within sensor readings 134), method 500 continues with step 586; otherwise, method 500 continues with block 588. In block 586, controller 116 sets target fan speed from the queue. Block 588 is a decision. If, in block 588, controller 116 determines that the current fan speed set point is equal to the target, method 500 continues with block 590; otherwise, method 500 continues with block 592. In block 590, controller 116 increases or decreases the current fan speed set point according to the target fan speed as determined in block 516. In block 592, controller 116 waits one second (or other predetermined time period) before continuing with block 584 (or other blocks within method 500). Blocks 584 through 592 thus repeat at a defined interval.

In block 596, controller 116 waits to receive a fan RPM signal interrupt from fan controller 126 (or directly from ventilation fan 114). In block 597, controller 116 increments a fan revolution count in its memory. Block 598 is a decision. If, in block 598, controller 116 determines that 60 iterations (more or fewer iterations may be used without departing from the scope hereof) of the fan revolution count have been stored in its memory, method 500 continues with step 599; otherwise, method 500 continues with block 596 (or some other block in method 500). In block 598, controller 116 sends fan revolution count to the RPM queue and method 500 continues with block 596.

Any portion, portions, or combination of blocks, of method 500 may be included or not included in the method 500 without departing from the scope hereof. Furthermore, aspects of method 500 may be performed in parallel. For example, the blocks 554-562 may be performed in parallel with the blocks 564-572, and also or alternatively in parallel with the blocks 574-582, and also or alternatively in parallel with the blocks 584-592.

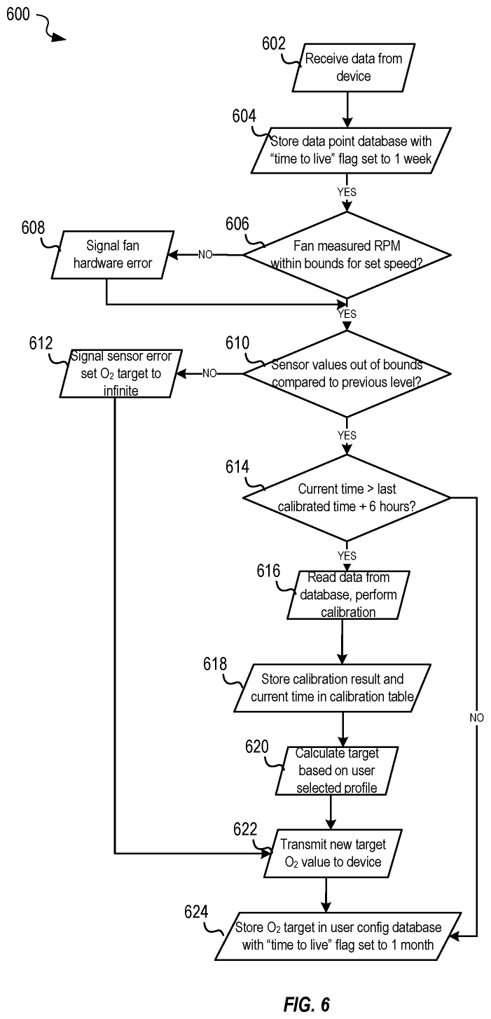

FIG. 6 is a flowchart illustrating one example process control method 600 performed by hypoxic level controller 136 of altitude simulator 100. Method 600 is implemented as computer-readable instructions that, when executed by one or more processors, cause the executing processor to implement the functionality of the hypoxic level controller 136 described herein.

In block 602, external server 130 receives data from controller 116. For example, block 602 may correspond to any block within method 500 where controller 116 transmits data to external server 130. In block 604, external server 130 stores data point databases with “time to live” flag set to 1 week (or some other period). Block 606 is a decision. If, in block 606, external server 130 determines that the measured fan RPM is within bounds for set speeds, then method 600 continues with block 610; otherwise, method continues with block 608.

In block 608, external server 130 generates a fan hardware error message. Method 600 then continues with block 610.

Block 610 is a decision. If, in block 610, external server 130 determine that sensor values are out of bounds compared to a previous level, method 600 continues with block 614; otherwise, method continues with block 612.

In block 612, external server 130 generates a signal sensor error and sets O₂ target to infinite to protect the user in case of a sensor problem. Block 614 is a decision. If, in block 614, external server 130 determines that the time interval between O₂ sensor calibrations has exceeded 6 hours (or some other period), method 600 continues with block 616; otherwise, method continues with block 624.

In block 616, external server 130 reads data from database and calculates O₂ sensor calibration based on current temperature, humidity, and barometric pressure inputs. In block 618, external server 130 stores calibration result and current time in a calibration table. In block 620, external server 130 calculates a target O₂ value based on user-selected altitude profile (for example the profile may include a “relaxed” profile increment of 1,000 ft per day, “normal” profile of 2,000 ft per day, “accelerated” profile of 3,000 ft per day, or a “manually input” user-defined altitude setting via the mobile application 144). In block 622, external server 130 transmits the new target O₂ value to controller 116.

In block 624, external server 130 stores the O₂ target in user config database with “time to live” flag set to one month (or some other period). Method 600 then ends.

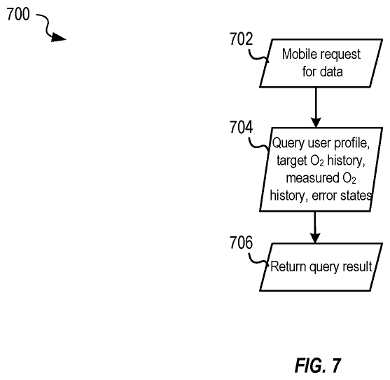

FIG. 7 is a flowchart illustrating one example method 700 for requesting data from external server 130 by the user device 142. Method 700 is implemented as computer-readable instructions that, when executed by one or more processors, cause the executing processor to implement the functionality between the external server 130 and the user device 142 described herein.

In block 702, mobile application 144 transmits a request for data to external server 130. In block 704, external server 130 queries the database at external server 130 for user profile, target O₂ history, measured O₂ history, and/or error states. In block 706, external server 130 sends the query results to mobile application 144.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. An altitude simulator for automatically controlling oxygen percentage in an enclosed environment, comprising:
a tent defining the enclosed environment and having an inlet;
an air unit;
air tubing having a first end coupled to the air unit and a second end positioned at the inlet;
a ventilation fan; and
a controller having:
a communication module;
an array of environmental sensors;
a processor; and
a memory storing machine readable instructions that, when executed by the processor, cause the processor to:
determine, based on the array of environmental sensors, an oxygen percentage of air within the enclosed environment; and
control a speed and direction of the ventilator fan to adjust the oxygen percentage within the enclosed environment.

2. The altitude simulator of claim 1, further comprising a sound attenuator coupled to the second end of the air tubing and positioned within the tent.

3. The altitude simulator of claim 2, wherein the air unit outputs hypoxic air into the air tubing such that the hypoxic air is released into the enclosed environment via the air tubing and the sound attenuator.

4. The altitude simulator of claim 1, the controller further comprising machine readable instructions that cause the processor to control the ventilation fan to dilute the hypoxic air inside the tent with air from outside the tent to achieve a desired oxygen percentage level inside said tent that simulates altitude.

5. The altitude simulator of claim 1, further comprising a mobile application operable to run on a mobile device of a user of the altitude simulator to interact with the controller via wireless communication.

6. The altitude simulator of claim 5, further comprising an external server that is wirelessly connected to the mobile application and the communication module.

7. The altitude simulator of claim 6, further comprising a Wi-Fi enabled interlock connected between the air unit and a power supply, the Wi-Fi enabled interlock operating under control of the mobile application or the controller to connect and disconnect the air unit from the power supply.

8. The altitude simulator of claim 1, the tent further comprising an integrated fan pocket for holding the ventilation fan, the integrated fan pocket being formed of mesh to allow the ventilation fan to generate an airflow into or out of the tent through the integrated fan pocket.

9. The altitude simulator of claim 1, the tent further comprising an interior mesh pocket for holding the controller, the interior mesh pocket allowing air within the tent to flow through the controller.

10. A method for simulating altitude in an enclosed environment, comprising:
determining, within a controller of an altitude simulator, a set altitude to be simulated within the enclosed environment;
determining an oxygen percentage setpoint based upon the set altitude;
sensing, at intervals, environmental parameters of the enclosed environment;
determining, from the environmental parameters, an oxygen percentage value indicative of a percentage of oxygen in air of the enclosed environment;
feeding hypoxic air into the enclosed environment from an air unit; and
controlling a ventilation fan to maintain the oxygen percentage value at the oxygen percentage setpoint.

11. The method of claim 10, further comprising:
sensing, prior to use of the enclosed environment, initial environmental parameters of the enclosed environment; and
determining oxygen calibration based on normoxia and the initial environmental parameters; and
adjusting the oxygen percentage value based on the oxygen calibration.

12. The method of claim 10, further comprising determining the set altitude based on a current date and time and an altitude profile that defines a starting time, a starting altitude, an altitude increment, an increment period, and a maximum altitude.

13. The method of claim 10, further comprising sending the oxygen percentage value to one or both of a mobile device of a user and a server.
